(19) United States
(12) Patent Application Publication
(10) Pub. No.: US 2012/0330202 A1
(43) Pub. Date: Dec. 27, 2012

(54) Thermal Therapy Device

(76) Inventor: Jonathan Flick, Chardon, OH (US)

(21) Appl. No.: 13/529,588

(22) Filed: Jun. 21, 2012

Related U.S. Application Data
(60) Provisional application No. 61/499,537, filed on Jun. 21, 2011.

(51) Int. Cl. A61F 5/00

(52) U.S. Cl. 602/14

(57) ABSTRACT

A thermal treatment device is provided to perform thermal therapy treatment to a portion of a body. The thermal treatment device includes a harness and a bladder. The harness is configured to wrap around at least a portion of the body to secure the thermal treatment device. The bladder is coupled to an interior section of the harness and is filled with a temperature retaining fluid to provide either cold or heat therapy treatment.

### PRIORITY

This application claims priority to U.S. Provisional Patent Application Ser. No. 61/499,537, filed Jun. 21, 2011, entitled “U SHAPED SHOULDER COLD THERAPY DEVICE,” the disclosure of which is incorporated by reference herein.

### BACKGROUND

The generally accepted first aid treatment for strains, sprains, contusions, dislocations, uncomplicated fractures, and other non-acute injuries to human limbs is often referred to by the acronym R.I.C.E., which stands for rest, ice, compression and elevation. Rest is prescribed because continued activity can cause further injury to the limb thereby delaying the healing process, increasing pain, and stimulating internal bleeding. Ice is prescribed because blood vessels contract when exposed to cold temperatures, which helps reduce swelling in the injured limb and also helps reduce the extent of internal bleeding from injured capillaries and blood vessels. In other instances, it may be desired to provide heat therapy to injured body areas. Compression is prescribed because it tends to hasten healing time by reducing swelling around the injury. And, elevation of the limb above the heart is prescribed because it tends to reduce swelling in the limb and also tends to reduce pain.

Because ice is a rigid non-conforming solid, alternative cold treatments may be desired over ice. Other cold therapy devices are known, as disclosed in U.S. Pat. No. 7,060,086, entitled “Thermal Treatment Pack,” issued Jun. 13, 2006; U.S. Pat. No. 7,481,786, entitled “Device for Administering Cold Therapy to Ankles,” issued Jan. 27, 2009; and U.S. Pat. No. 7,621,944, entitled “Thermal Pack,” issued Nov. 24, 2009, which patents are incorporated by reference herein.

While several devices and methods have been made and used for providing cold therapy treatment, it is believed that no one prior to the inventor has made or used the invention described in the appended claims.

### BRIEF DESCRIPTION OF THE DRAWINGS

While the specification concludes with claims which particularly point out and distinctly claim the invention, it is believed the present invention will be better understood from the following description of certain examples taken in conjunction with the accompanying drawings, in which like reference numerals identify the same elements and in which:

FIG. 1 depicts a perspective view of an exemplary thermal therapy device;

FIG. 2 depicts a front view of the exemplary thermal therapy device of FIG. 1;

FIG. 3 depicts a side view of the exemplary thermal therapy device of FIG. 1;

FIG. 4 depicts a top view of the exemplary thermal therapy device of FIG. 1;

FIG. 5 depicts a perspective view of an exemplary harness of the exemplary thermal therapy device of FIG. 1;

FIG. 6 depicts a front view of another exemplary thermal therapy device;

FIG. 7 depicts an exploded view of the exemplary thermal therapy device of FIG. 6;

FIG. 8 depicts a side view of an exemplary bladder of the exemplary thermal therapy device of FIG. 1;

FIG. 9 depicts a top view of the exemplary bladder of FIG. 8;

FIG. 10 depicts an elevation view of the exemplary thermal therapy device of FIG. 1 with the exemplary bladder being positioned onto the exemplary harness;

FIG. 11 depicts a perspective view of the exemplary thermal therapy device of FIG. 1 positioned on a shoulder; and

FIG. 12 depicts a perspective view of the exemplary thermal therapy device of FIG. 1 positioned on a hip.

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the invention should not be used to limit the scope of the present invention. Other examples, features, aspects, embodiments, and advantages of the invention will become apparent to one skilled in the art from the following description. As will be realized, the invention is capable of other different and obvious aspects, all without departing from the invention. Accordingly, the drawings and descriptions should be regarded as illustrative in nature and not restrictive.

I. Exemplary Thermal Therapy Device

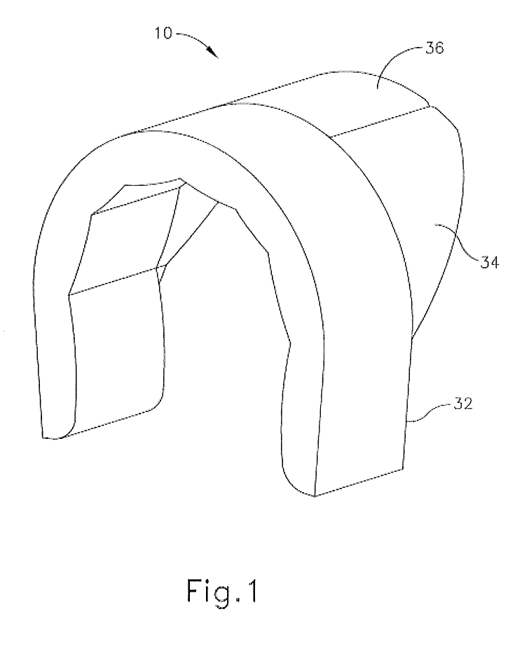

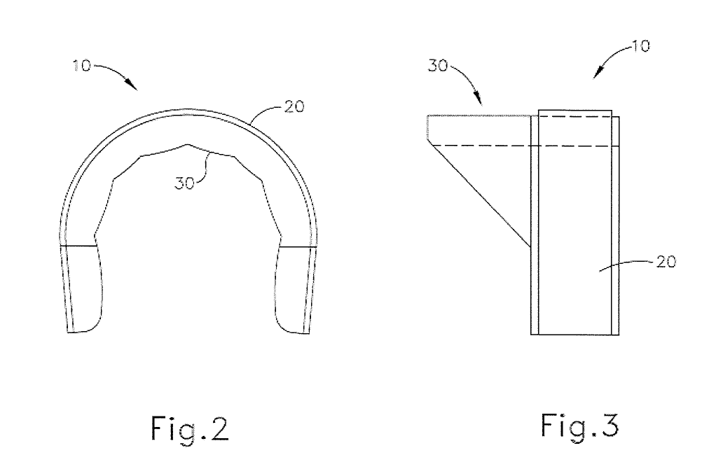

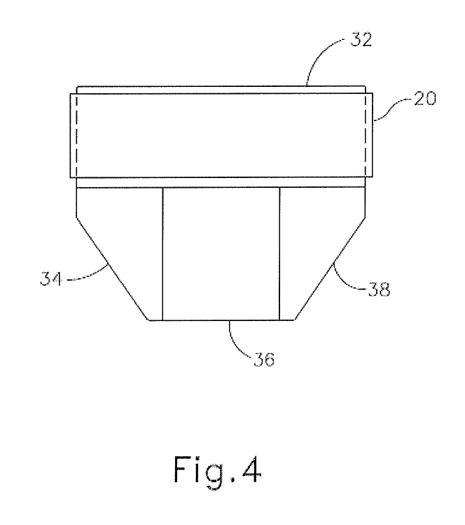

An exemplary thermal therapy device (10) is shown in FIGS. 1-4. Device (10) comprises a bladder (30) and a backbone harness (20). Bladder (30) may be filled with a fluid that is chilled or heated to provide thermal therapy to a user. Bladder (30) may be positioned around harness (20) such that bladder (30) is positioned on the interior side (22) of harness (20), as shown in FIG. 2. Accordingly, bladder (30) and harness (20) may be placed on a user such that bladder (30) contacts the user to provide thermal therapy and harness (20) holds bladder (30) in position. A portion of bladder (30) may also extend outwardly past the surface of harness (20) on one or both sides of harness (20), as shown in FIG. 3. In the present example, device (10) is shaped as an inverted U, which may be sized to fit the shoulder of a user. Device (10) may also be sized to fit other body parts, such as the hip, arm, finger, wrist, neck, forehead, head, rib cage, waist, thigh area, knee, leg, calf, foot, etc. Other suitable configurations will be apparent to one with ordinary skill in the art in view of the teachings herein.

A. Exemplary Harness

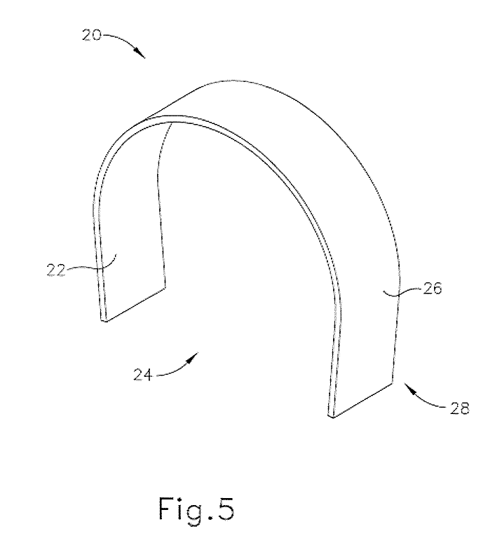

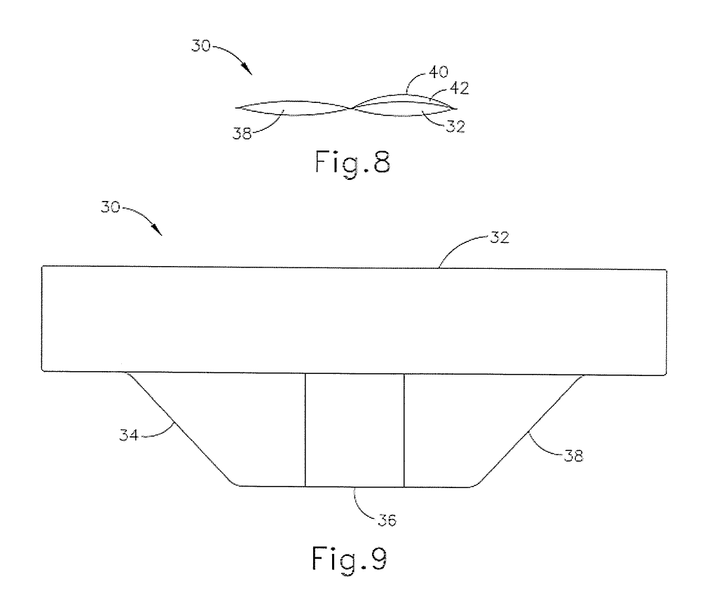

As shown in FIG. 5, harness (20) may comprise a band that is shaped as an inverted U. Harness (20) comprises an interior side (22), an exterior side (26), ends (28), and opening (24). Interior side (22) is the underneath portion of U-shaped harness (20) and is configured to face the user and be coupled to bladder (30). Exterior side (26) faces outwardly from the user when harness (20) is positioned on the user. Ends (28) are located on each side of the U on harness (20). Ends (28) may project inwardly to contact the user and securely hold thermal therapy device (10) to the user. Opening (24) of harness (20) is located between ends (28) to provide area for the patient to insert an injured area into thermal therapy device (10). The inverted U shape of harness (20) may be sized to be placed around a variety of body parts, including a shoulder. Harness (20) may be placed on a user such that harness (20) wraps around the front, top, and back portion of the user's shoulder (FIG. 8). Once placed, harness (20) may retain the inverted U shape to fit securely to a user's shoulder. The secure fit of harness (20) to a user's shoulder will act to contact bladder (30) to the user's shoulder and hold bladder (30) in place for thermal therapy treatment.

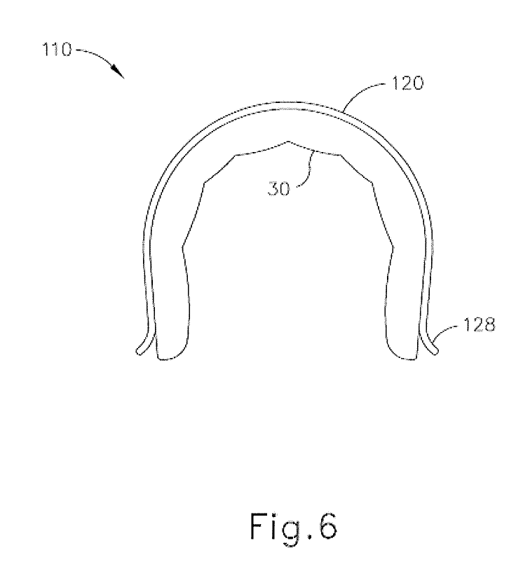

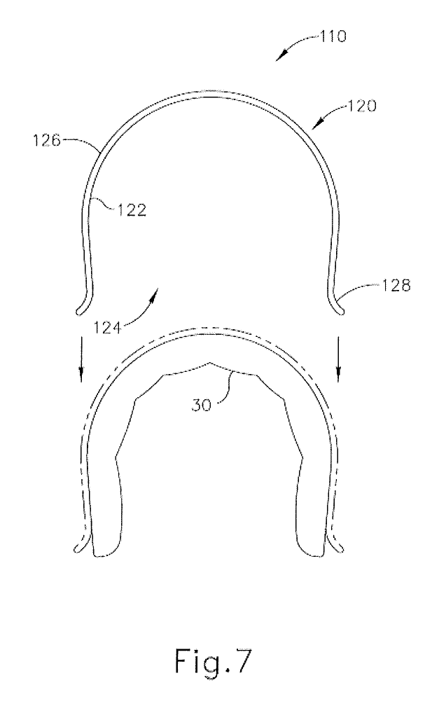

FIGS. 6 and 7 depict another exemplary harness (120). Harness (120) is similar to harness (20), except that harness (120) includes flared ends (128). Flared ends (128) extend outwardly from the inverted U shape of harness (120). Flared ends (128) may secure bladder (30) to harness (120) such that flared ends (128) may prevent bladder (30) from slipping off of harness (120). Other suitable harness variations to hold bladder (30) in place will be apparent to one with ordinary skill in the art in view of the teachings herein.

Harness (20, 120) may also provide a slight compression on the user, which is part of the R.I.C.E. cold therapy method. Harness (20, 120) may have resilient properties such that the ends (28, 128) of harness (20, 120) are able to flex outwardly to place harness (20, 120) onto the injured body area. The ends (28, 128) of harness (20, 120) may then be resiliently biased inward in order to contact the injured body area and provide a secure fit of cold therapy device (10, 110) on the user. Accordingly, a user may benefit from compression and thermal therapy treatment by a resiliently biased harness (20, 120) of thermal therapy device (10, 110).

Harness (20, 120) may be formed from a metal, a composite, or a plastic material. For instance, harness (20, 120) in the present example is formed from a clear polyvinyl chloride (PVC), Harness (20, 120) may be thick enough in order to substantially retain the inverted U shape. Harness (20, 120) may also be thin enough to be formed into the inverted U shape. In the present example, harness (20, 120) is about 3/16 inches thick. The band of desired material may be heated and shaped into the desired configuration, such as an inverted U, to form harness (20, 120). Other suitable materials and thicknesses will be apparent to one with ordinary skill in the art in view of the teachings herein.

B. Exemplary Bladder

An exemplary bladder (30) is shown in FIGS. 8 and 9. Bladder (30) may be divided into chambers to disperse and retain a fluid throughout bladder (30) to provide substantially even thermal therapy about the user's injured area. In the present example, bladder (30) comprises chambers (32, 34, 36, 38), top layer (40), and pocket (42). Interior chamber (32) is configured to be positioned on harness (20, 120). Interior chamber (32) may be sized substantially to the length of harness (20, 120) to be positioned along the interior side (22, 122) of harness (20, 120). Interior chamber (32) is flexible and allows bladder (30) to take the shape of the inverted U of harness (20, 120) when bladder (30) is coupled to harness (20, 120).

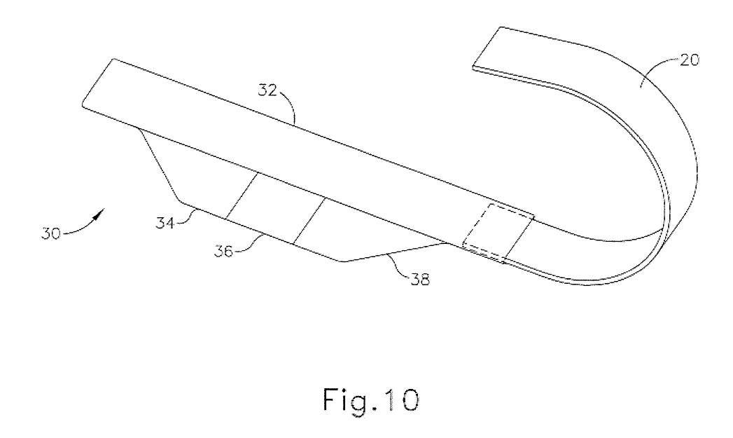

Interior chamber (32) may be secured to harness (20, 120) to hold bladder (30) onto harness (20, 120). For instance, interior chamber (32) comprises a top layer (40) of film on the top surface of interior chamber (32) to form a pocket (42). Harness (20, 120) may then be slid into pocket (42) on top of interior chamber (32) to couple harness (20, 120) to bladder (30) (FIG. 10). When placed into pocket (42), interior side (22, 122) of harness (20, 120) may contact the top surface of interior chamber (32), while exterior side (26, 126) may contact top layer (40). Interior chamber (32) is then positioned on interior side (22, 122) of harness (20, 120) to contact the user when thermal therapy device (10, 110) is positioned on the injured area. Top layer (40) of interior chamber (32) wraps around exterior side (26, 126) of harness (20, 120) to secure harness (20, 120) to bladder (30). Harness (20, 120) may also be secured to interior chamber (32) by an adhesive. Alternatively, harness (20, 120) may be placed inside of interior chamber (32) such that harness (20, 120) is sealed into place within interior chamber (32). Other suitable methods of securing harness (20, 120) to bladder (30) will be apparent to one with ordinary skill in the art in view of the teachings herein.

Once coupled, harness (20, 120) and bladder (30) may be positioned on a user such that harness (20, 120) provides a secure fit to the user and bladder (30) contacts the user to provide thermal therapy to the user. For instance, thermal therapy device (10, 110) may be placed on a user's shoulder. Interior chamber (32) may then wrap around the front, top, and back shoulder areas of a user when thermal therapy device (10, 110) is placed on the user. Thermal therapy from interior chamber (32) may be focused on a variety of shoulder muscles, such as the supraspinatus muscle, infraspinatus muscle, teres minor muscle, subscapularis muscle, etc, or other joint areas.

Bladder (30) further comprises exterior chambers (34, 36, 38) that extend from interior chamber (32). In one embodiment, chamber (36) is rectangular and extends from the central area of interior chamber (32). Accordingly, chamber (36) may be positioned on the top of a user's shoulder during use of device (10, 110). Chambers (34, 38) are trapezoidal and extend from interior chamber (32) in each side of chamber (36). The trapezoidal shape of chambers (34, 38) allow bladder (30) to contour to the user's body shape, such as the shoulder. Accordingly, chambers (34, 38) may be positioned on the front and back of the user's shoulder area during use of device (10, 110). Chambers (34, 36, 38) may apply thermal therapy to the rotator cuff area.

Bladder (30) may consist of multiple chambers with various shapes and sizes to accommodate different shoulder thermal therapy applications. For instance, thermal therapy device (10, 110) may be placed on a user to treat an injury to the hip, arm, finger, wrist, neck, forehead, head, rib cage, waist, thigh area, knee, leg, calf, foot, etc. While the present example is configured for a shoulder, chambers (32, 34, 36, 38) of bladder (30) may be adjusted to a smaller size to accommodate a smaller body area, or chambers (32, 34, 36, 38) may adjusted to a larger size to accommodate a larger body area. Further, the amount of chambers (32, 34, 36, 38) of bladder (30) may decrease to accommodate a smaller body area or increase to accommodate a larger body area. Other suitable chamber configurations will be apparent to one with ordinary skill in the art in view of the teachings herein.

As disclosed in U.S. Pat. Nos. 7,060,086, 7,481,786, and 7,621,944, which patents are incorporated by reference above, bladder (30) may be formed by a continuous film of flexible material. The film of bladder (30) may be heat sealed to itself, which facilitates in forming chambers (32, 34, 36, 38) of bladder (30). Further, the film of bladder (30) may be made of a material that does not irritate human skin. The continuous film may be a polymeric film that remains flexible over a relatively broad temperature range of from about 0° F. to about 130° F. The polymeric film may be a single layer or a multi-layer structure. In one embodiment, the continuous film may exhibit a Shore A durometer hardness of greater than or equal to 85 as measured in accordance with the ASTM 2240.00 standard. The continuous film may also exhibit a Shore A durometer hardness of 93 +/−5 as measured in accordance with the ASTM 2240.00 standard. The continuous film of bladder (30) may have a thickness from about 3 to about 15 mils. The continuous film may also have a thickness of 5 +/−2 mils. Films possessing such properties may be puncture resistant, yet remain flexible. Other suitable film characteristics may be apparent to one with ordinary skill in the art in view of the teachings herein.

As disclosed in U.S. Pat. Nos. 7,060,086, 7,481,786, and 7,621,944, which patents are incorporated by reference above, a fluid is received into chambers (32, 34, 36, 38) of bladder (30). The fluid may be a thermal fluid that may be heated or cooled. The fluid may also retain the heated or cooled temperature for a duration of time after the fluid has been exposed to the heat or cold. The temperature retaining fluid may be any non-gaseous material that changes shape or direction uniformly in response to an external force imposed upon it and that has the capacity to retain heat or cold for transfer to a limb. The term applies to liquids and finely divided solids, gels and combinations of liquid and solid particles having such properties. The temperature retaining fluid may be a water-based gel formulated to provide a fluid that is more viscous than water and does not become a hard solid within the temperature range of from 0° F. to 32° F. Such gels can conform to the contours of the injured limb more comfortably than ice. In addition, such gels tend to retain their desired thermal properties for a longer period of time than water. The gel filled chambers (32, 34, 36, 38) of bladder (30) may be pre-heated or pre-chilled, depending upon the type of thermal treatment to be provided. The temperature retaining fluid may be a non-toxic formulation to minimize potential injury in the event of exposure.

In another example, water-filled capsules are dispersed in the temperature retaining fluid. When bladder (30) is pre-chilled (e.g., through storage in a conventional freezer), the water in the water-filled capsules freezes and becomes a solid, although the gel remains a viscous fluid. The frozen water-filled capsules extend the period of time during which bladder (30) remains cold. The water-filled capsules may be small and substantially spherical to allow bladder (30) to comfortably conform to limbs. Other free-flowing solid structures can be dispersed in the temperature-retaining fluid, such as inorganic particulates (e.g., sand, ceramic particles, etc.), spherical structures (glass, metal, etc.), magnets, etc. Other suitable substantially free-flowing structures may be dispersed within chambers (32, 34, 36, 38) as will be apparent to one with ordinary skill in the art in view of the teachings herein.

It may be desirable to dispose an insulating layer between the limb and bladder (30) to prevent frostbite if bladder (30) has been pre-chilled. The insulation layer may be formed of fabric or polymeric foam. The insulation layer may be wrapped around the limb prior to applying device (10) (e.g., a sleeve, sock, ACE bandage, etc.), or the insulation layer may be disposed as a layer around bladder (30) so as to be coextensive with at least the inner surface of bladder (30).

II. Exemplary Application

Thermal therapy device (10, 110) may be chilled or heated and applied to a portion of the human body to provide thermal therapy treatment. Bladder (30) may be chilled by storing bladder (30) in a freezer, refrigerator, cooler, etc. Alternatively, bladder (30) may be heated by warming water and placing bladder (30) in the water. Other suitable cooling or heating methods will be apparent to one with ordinary skill in the art in view of the teachings herein. Once bladder (30) reaches a desired temperature, bladder (30) may be coupled to harness (20, 120). Alternatively, bladder (30) and harness (20, 120) may be coupled and then both bladder (30) and harness (20, 120) may be chilled or heated while they are coupled. In the present example, harness (20, 120) may be coupled to bladder (30) by sliding harness (20, 120) into pocket (42) on interior chamber (32) of bladder (30) (FIG. 10). Accordingly, harness (20, 120) may be slid into and secured to interior chamber (32) between top layer (40) of film attached to the top surface of interior chamber (32) and interior chamber (32). The temperature retaining fluid within interior chamber (32) may be positioned on interior side (22, 122) of harness (20, 120). Once harness (20, 120) is fully positioned within pocket (42) on interior chamber (32), bladder (30) conforms to the shape of harness (20, 120) (FIG. 1).

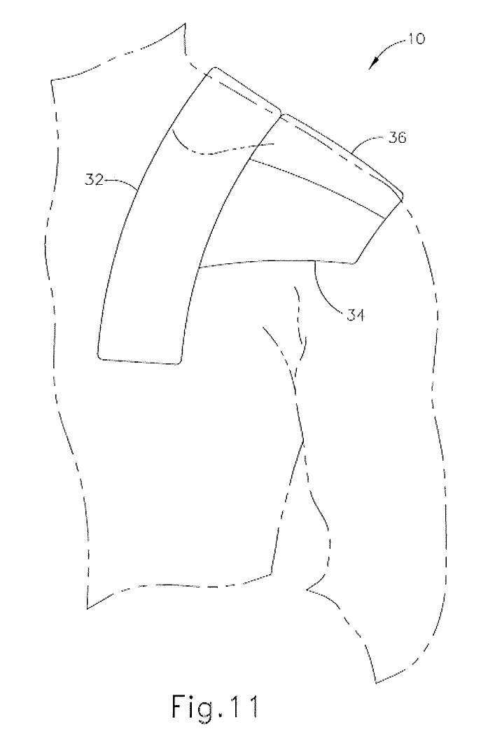

Once harness (20, 120) and bladder (30) are coupled, thermal therapy device (10, 110) may be applied to the desired body area for treatment. In the present example, thermal therapy device (10, 110) may be applied to the shoulder area. However, thermal therapy device (10, 110) may be applied to other body parts such as the hip, arm, finger, wrist, neck, forehead, head, rib cage, waist, thigh area, knee, leg, calf, foot, etc. Thermal therapy device (10, 110) may be placed on a user's shoulder as shown in FIG. 11. A user may pick up thermal therapy device (10, 110) such that opening (24, 124) of the inverted U shape of harness (20, 120) is positioned over the shoulder. The user may then slide thermal therapy device (10, 110) down and onto the shoulder. The user may grasp harness (20, 120) to position thermal therapy device (10, 110) onto the shoulder with one hand. This may be desirable if the user has an injury that immobilizes the shoulder and arm, leaving the user with only one functioning arm to place thermal therapy device (10, 110). Ends (28, 128) of the inverted U shaped harness (20, 120) may flex outwardly as thermal therapy device (10, 110) is placed on the shoulder. Harness (20, 120) may then be resiliently biased inward toward the shoulder area to snugly fit harness (20, 120) to the shoulder and hold thermal therapy device (10, 110) in place. The resilient bias of harness (20, 120) may also provide compression treatment to the shoulder. Interior chamber (32) of bladder (30) may be positioned on the interior side (22, 122) of harness (20, 120) to contact the shoulder area. Harness (20, 120) and interior chamber (32) of thermal therapy device (10, 110) wrap around the front, top, and back portions of the user's shoulder. Chambers (34, 36, 38) may then extend outwardly from interior chamber (32) to provide more coverage of thermal therapy to the shoulder.

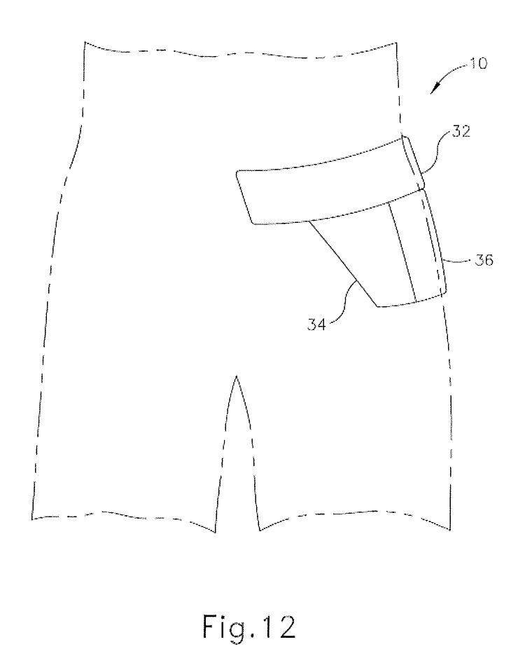

As mentioned above, thermal therapy device (10, 110) may be applied to treat a hip injury, as shown in FIG. 12. The inverted U shape of harness (20, 120) may slip around and cradle the hip joint area in such a manner that would deliver thermal therapy. The sizing and fit of harness (20, 120) may be adjusted accordingly to fit hips. The number of chambers of bladder (30) may also be increased, such as to eight, to accommodate a waist size. Alternatively, bladder (30) may be placed around a belt or a strap and positioned around the waist of a user to hold thermal therapy device (10, 110) in place. Thermal therapy device (10, 110) may also be configured in a number of sizes to fit other body areas, (arm, finger, wrist, neck, forehead, head, rib cage, waist, thigh, knee, leg, calf, foot, etc.) by adjusting the size of harness (20, 120) and corresponding bladder (30). Other applications of thermal therapy device (10, 110) will be apparent to one with ordinary skill in the art in view of the teachings herein.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometries, materials, dimensions, ratios, steps, and the like discussed above are illustrative. Accordingly, the scope of the present invention should be considered in terms of the following claims and is understood not to be limited to the details of structure and operation shown and described in the specification and drawings.

I/we claim:

1. A thermal treatment device comprising:
(a) a harness, wherein the harness comprises an interior section, and wherein the harness is configured to wrap around at least a portion of a body; and
(b) a bladder coupled to the harness, wherein the bladder comprises at least one chamber, wherein the at least one chamber is configured to be positioned on the interior section of the harness, and wherein the at least one chamber is filled with a fluid.

2. The thermal treatment device of claim 1, wherein the harness is U-shaped.

3. The thermal treatment device of claim 1, wherein the harness is resiliently biased toward the portion of the body that the harness is wrapped around.

4. The thermal treatment device of claim 1, wherein the harness is made from a metal, a composite, or a plastic.

5. The thermal treatment device of claim 4, wherein the harness is made from polyvinyl chloride.

6. The thermal treatment device of claim 1, wherein the harness is wrapped around at least a portion of a shoulder area.

7. The thermal treatment device of claim 1, wherein the harness is wrapped around at least a portion of a hip area.

8. The thermal treatment device of claim 1, wherein the bladder further comprises a coupling feature disposed on a top surface of the at least one chamber, wherein the coupling feature is configured to couple the harness to the bladder.

9. The thermal treatment device of claim 8, wherein the coupling device feature is configured to removably couple the harness to the bladder.

10. The thermal treatment device of claim 9, wherein the coupling feature is a pocket.

11. The thermal treatment device of claim 1, wherein the bladder further comprises a main chamber coupled to the harness and a plurality of other chambers extending from the main chamber.

12. The thermal treatment device of claim 11, wherein three chambers extend from the main chamber, wherein a substantially rectangular chamber extends from a central portion of the main chamber, and wherein two substantially trapezoidal chambers extend from the main chamber on each side of the substantially rectangular chamber.

13. The thermal treatment device of claim 1, wherein the fluid is a temperature retaining fluid, wherein the temperature retaining fluid is capable of being one of chilled or heated, wherein the temperature retaining fluid is capable of retaining the chilled or heated temperature for a period of time after the temperature retaining fluid has been chilled or heated.

14. The thermal treatment device of claim 13, wherein the temperature retaining fluid is a water-based gel.

15. The thermal treatment device of claim 1, wherein the bladder is made from a polymeric film.

16. The thermal treatment device of claim 1, wherein the bladder further comprises an insulating layer.

17. A thermal treatment device comprising:
(a) a U-shaped harness, wherein the harness comprises an interior section, and wherein the harness is configured to wrap around at least a portion of a body; and
(b) a bladder, wherein the bladder comprises:
(i) a main chamber filled with a temperature retaining fluid,
(ii) at least one other chamber extending from the main chamber, wherein the at least one other chamber is filled with a temperature retaining fluid, and
(iii) a coupling feature disposed on a top surface of the main chamber, wherein the pocket is configured to couple the main chamber to the interior section of the harness.

18. A method of using a thermal therapy device to provide thermal treatment to at least a portion of a body, wherein the thermal therapy device comprises a U-shaped harness having an interior section and a bladder comprising at least one chamber filled with a temperature retaining fluid, the method comprising:
(a) chilling or heating the bladder;
(b) coupling the bladder to the interior section of the U-shaped harness;
(c) positioning an opening of the U-shaped harness relative to a portion of the body to be treated; and
(d) positioning the U-shaped harness and bladder around the portion of the body to be treated.

19. The method of claim 18, wherein the thermal therapy device is positioned around a shoulder area.

20. The method of claim 18, wherein the thermal therapy device is positioned around the body area using one hand.
